(19) United States
(12) Patent Application Publication
(10) Pub. No.: US 2023/0189894 A1
(43) Pub. Date: Jun. 22, 2023

(54) INHALER

(71) Applicant: KÖRBER TECHNOLOGIES GMBH, HAMBURG (DE)

(72) Inventors: ALP OHRI, HAMBURG (DE); MARC KESSLER, HAMBURG (DE); CHRISTIAN HANNEKEN, HAMBURG (DE)

(21) Appl. No.: 17/918,789

(22) PCT Filed: Apr. 13, 2021
(86) PCT No.: PCT/EP2021/059538

§ 371 Date: Oct. 13, 2022

(30) Foreign Application Priority Data
Apr. 15, 2020 (DE) 10 2020 110 258.5

(51) Int. Cl. A24F 40/53; A24F 40/10

(52) U.S. Cl. CPC A24F 40/53; A24F 40/10

(57) ABSTRACT

An inhaler comprises a housing, an air channel extending between at least one air inlet opening and a suction opening in the housing, a dispensing element for vaporizing or nebulizing liquid supplied to the dispensing element for admixing with air flowing in the air channel, an electronic control device, an electronic data memory, and a sensor system comprising a flow measuring device for measuring the volumetric and/or mass flow of air flowing through the air channel. The electronic control device is adapted to capture a plurality of airflow measurement values over at least a portion of the duration of an inhalation puff by means of the flow measurement device, compare the plurality of airflow measurement values to a puff profile stored in the data memory, and output a control signal based on the comparison of the plurality of airflow measurement values to the stored puff profile.

The present invention relates to an inhaler comprising a housing, an air channel extending between at least one air inlet opening and a suction opening in the housing, a dispensing element for nebulizing or vaporizing liquid supplied to the dispensing element for admixture with air flowing in the air channel, an electronic control device, an electronic data storage device, and a sensor system comprising a flow measuring device for measuring the volumetric and/or mass flow of the airflow flowing through the air channel.

Such an inhaler is known, for example, from EP 3 574 779 A2.

Today’s electronic cigarette products and inhalers dose the active ingredient to be delivered via a preset, user-independent delivery mechanism comprising a dispensing control and a dispensing element. The control activates the dispensing element, for example, as a result of a negative pressure measured by a sensor during an inhalation puff. The amount of active ingredient delivered is thereby essentially determined by the activation time of the dispensing element. A dispensing element may be a heating element, an ultrasonic nebulizer that vaporizes or nebulizes liquid by means of a piezo element, a gas compressor that builds up gas pressure and thereby nebulizes or vaporizes liquid through a nozzle, or a nebulizing membrane in which liquid is vaporized or nebulized by highfrequency oscillation of the membrane.

Since there is currently no correlation between the dispensing control and the user-specific air volume drawn through the inhaler during an inhalation puff (e.g., the amount of air drawn differs for a strong or weak inhalation draw), the vapor quality, i.e., the amount of active ingredient per air volume as well as the droplet size distribution, is essentially dependent on the user’s individual draw behavior. This can negatively influence both the reproducible smoking experience and a defined active ingredient delivery in terms of quantity and delivery timing into the inhaled air volume.

The task of the present invention is to provide an inhaler with improved reproducibility of both the smoking or vaping experience as well as the active ingredient dosage.

The invention solves this task with the features of the independent claims.

According to the invention, a capture of a plurality of airflow measurement values takes place over a part or the entire duration of an inhalation puff. Subsequently, a comparison of the plurality of airflow measurement values with a puff profile stored in the data memory takes place. A control signal is output based on the result of the comparison of the plurality of airflow measurement values to the stored puff profile. The stored puff profile can be viewed like a calibration. The air flow measurement values are air volume flow and/or air mass flow measurement values measured by the flow measurement device as a function of time and/or pressure changes in the area of the inhaler through which the air mass flow passes during the inhalation puff. A puff profile in the sense of the invention is a defined ideal time profile of an air or air-vapor volumetric flow (and/or mass flow) that flows through the inhaler during at least part or all of the user’s ideal inhalation puff. According to the invention, a comparison is made between the predetermined puff profile and measured values recorded during an actual inhalation puff.

In particular, the invention provides feedback based on the measured airflow profile during an inhalation puff, and preferably also the amount of active ingredient/liquid dispensed during the inhalation puff. Due to the preferred feedback between the airflow profile during an inhalation puff, and preferably also the amount of active ingredient/liquid dispensed during the inhalation puff, based on the result of the comparison with an ideal puff profile, a defined and reproducible dispensing of active ingredient or an indication to the user of an incorrect use of the inhaler is possible. Through the control according to the invention, the inhaler virtually adapts to a user by taking into account the individual breath of a user during the dispensing of active ingredient or liquid into the airflow.

Alternatively, it is conceivable that the inhaler comprises a fixed active ingredient dispensing profile and the user is trained to maintain a matching airflow, i.e., inhalation profile, by means of a corresponding feedback loop, in which deviations are signaled to the user accordingly.

Preferably, in the event of a defined deviation of the airflow measurement values from the stored puff profile, the control signal triggers a suitable action. The suitable action preferably comprises adjusting the control of the heating element (more precisely the heating current flowing through the heating element and/or the heating duration and/or a pulse-pause ratio of the control), adjusting the vibration speed of a piezo element or a nebulizing membrane, or adjusting the exit speed of a compressed gas from a nozzle by adjusting a gas pressure, e.g. the air pressure for the amount of liquid to be administered, and/or signaling a corresponding information to a user on a signaling device.

Accordingly, based on the result of the comparison between the flow measurement values and the puff profile, various alternative responses can be made. A preferred option is to signal to the user of the inhaler by means of a suitable signaling device that, for example, the amount of active ingredient dispensed during the inhalation puff was insufficient. In the scenario of too strong an inhalation puff and a possible associated overdose, the administration can be interrupted with the aid of the electronic control system so that the occurrence of medication side effects can be avoided or, in an emergency, appropriate measures can be initiated (e.g. calling the emergency doctor or ambulance).

Another preferred option is that, based on the degree of deviation of the measured values from the puff profile, the active ingredient dispensing into the air/air vapor volume flow is adjusted by appropriate control of the dispensing device or the heating element, the piezo element, the nebulization membrane or the air compressor. This is done by adjusting the heating current flowing through the heating element based on the control signal. Advantageously, if the heater current is controlled using digital pulse width modulation (PWM) with two discrete states, one or more of the following parameters can be used to adjust the heater current: duration of the ON state (voltage applied); duration of the OFF state (no voltage applied); the ratio between the duration of the ON state and duration of the OFF state (pulse-pause ratio); other activation/deactivation limits, such as resistance limits of the heater; magnitude of the level, i.e., of the voltage applied to the heating element, during the ON state.

It is also possible that both of the previously described reactions are performed. For example, an attempt may first be made to adjust the amount of active ingredient delivered by vaporization control, i.e., adjusting the control of the heating element or the heating current flowing through the heating element. If this is no longer possible during the respective inhalation puff, e.g. due to an excessive deviation of the measured values from the puff profile, the deviation from the puff profile can be signaled to the user at the end and/or after the inhalation puff in such a way that the user can adjust his inhalation puff. If, in turn, such an adjustment of the inhalation puff is not sufficient, the user can be signaled an incorrect dosage.

In accordance with the invention, the preferred feedback can advantageously be implemented by a suitable control circuit, in particular comprising a digital electronic control device (for example, microprocessor), an electronic, in particular non-volatile data memory and a sensor system or part thereof. The electronic data memory is set up to permanently store at least one puff profile. The sensor system comprises a flow measuring device, which is arranged to measure the temporal course of the air/air-vapor volumetric flow during a specific inhalation puff. The electronic control device is expediently connected to the sensor system, the data storage device, the heating element and any signaling device that may be present.

The flow measuring device may advantageously be a differential pressure measuring device, which enables reliable measurement of the air volume flow (or air mass flow) with simple means in a mobile inhaler. In this embodiment, the differential pressure measurement device comprises a first pressure sensor arranged to measure the ambient air pressure of the inhaler and a second pressure sensor arranged to measure the air pressure in the air channel of the inhaler. By knowing the cross-section of the air channel at the measurement location of the second pressure sensor and the pressure difference measured between the first and second pressure sensors, the air flow rate through the inhaler can be determined.

In another embodiment, the flow measuring device may advantageously be a hot wire measuring device (thermal anemometer). In this case, at least one wire element arranged in the air channel of the inhaler is electrically heated. As a result of the flow around the wire element, heat is transported into the flowing air, i.e. the wire element is cooled. By measuring the electrical resistance, which depends on the temperature, it is thus possible to determine the flow velocity, and thus the volumetric flow rate, of the air flowing through the air channel.

By means of the flow measuring device, it is also possible to determine the air volume flow over time. This advantageously results in a “puff” profile or measured puff profile of the entire puff.

Preferably, the sensor system comprises a liquid volume sensor for capturing the amount of liquid dispensed by a vaporization device during an inhalation puff. This allows a more precise adaptation of the amount of liquid to be vaporized to the respective puff profile of the consumer.

In an advantageous embodiment, the liquid amount sensor is an air humidity sensor. Preferably, the humidity sensor comprises two humidity measuring elements arranged in the air channel of the inhaler for measuring the humidity of the air. The first humidity measuring element is arranged in the area of the air inlet or in the air channel upstream of the heating element and measures a reference value of the air humidity before the admixture of liquid vapor. The second humidity measuring element is located in the air channel downstream from the heating element, for example in the area of the mouthpiece, and measures the humidity of the air directly before the user inhales the air-aerosol-vapor mixture. Given a known amount of water in the liquid to be vaporized and a calibration of the second humidity measuring element, the amount of liquid vaporized can be determined from the increase in humidity, i.e. the difference between the measured values from the second and the first humidity measuring element, and taken into account in the further process.

A changing water content in the liquid due to differential distillation during the emptying of the cartridge can advantageously be compensated by determining the residual liquid quantity in the liquid tank by means of a level sensor (see below) or estimation by counting the puffs in connection with the measurement of the puff duration.

From the determined residual liquid quantity, the current water content can be calculated on the basis of knowledge of the differential distillation, and the measured value of the humidity can be corrected on this basis. In addition, the vaporized amount of active ingredient can be determined from the determined or estimated vaporized amount of liquid, taking into account the proportion of active ingredient in the liquid.

Advantageously, the sensor system comprises a level sensor for capturing a residual liquid amount in a liquid tank of the inhaler. The level sensor can be a capacitive sensor, for example. From the level of liquid in the liquid tank measured by the level sensor, the residual amount of liquid in the liquid tank can be easily determined. The electronic control device is preferably adapted to determine a duration until a cartridge change is required based on the amount of remaining liquid measured by means of the level sensor, for example taking into account a previous usage profile stored in the data memory.

Alternatively, the level sensor can be based on an absorption measurement. For this purpose, a light source with a defined wavelength, such as an LED or a laser, and a light-sensitive element (photoelement), such as a photodiode, are arranged in or with respect to the air channel in such a way that the airflow passing through the air channel flows through the area between the light source and the light-sensitive element. The wavelength of the light emitted by the light source is selected in such a way that the active ingredient vaporized into the gas phase by the heater comprises a high absorption cross-section at this wavelength specific to the active ingredient (e.g. in the IR or UV range). As the air enriched with the active ingredient passes through, the photoelement measures the absorption (attenuation) of the light emitted by the light source. The measured absorption value over time, in conjunction with the determination of the airflow rate through the air channel, is proportional to the amount of active ingredient vaporized into the airflow.

The signaling device may include, for example, one or more LEDs, a digital display, a display, a haptic signaling device, and/or an audible signaling device. The electronic control device is arranged to compare the measured values of the sensor system, more specifically the flow measuring device, with the at least one puff profile. If the result of the comparison is a deviation between the measured values and the puff profile that exceeds a certain threshold value, the electronic control device is arranged to control the heating element and/or the signaling device in such a way that a predetermined reaction takes place depending on the determined deviation. A predetermined response may be, for example, an increase or decrease of the heating time of the heating element to change the vaporization respectively a change in the pulse-to-pause ratio and thus the delivery of the active ingredient into the air/air-vapor volume flow. For example, a predetermined response may also be an instruction to the user, indicated by signaling device, to adjust their inhalation puff. Alternatively, the user may be indicated that an incorrect dosage of the active ingredient has occurred.

In particular for the application of the invention in the medical sector, the puff profile can be determined in advance in an initialization procedure, for example as a function of a series of inhalation puffs of a patient, i.e. a user- or patient-specific puff profile is determined. The initialization procedure described below is not limited to medical applications, but can also be used in non-medical applications, for example stimulant applications.

For this purpose, several inhalation puffs without active ingredient dispensing by vaporizing at the heating element can be measured before the actual application (“dry puffs”). It is also conceivable that one or more breaths could be measured, e.g. by lung volume and/or function measurements. Such measurements could, for example, be performed externally by a physician or with the inhaler itself. Externally recorded data can be transmitted via a communication device of the inhaler (e.g., wirelessly based on, for example, Bluetooth, WLAN, RFID, ZigBee, optical, or wired) to the electronic control device and/or the data storage device. By comparing this with a theoretical puff profile, the patient’s individual puff profile can be determined. This (patient) individual puff profile is stored in the electronic memory. During the actual use of the inhaler with active ingredient dispensing, the electronic control device compares this (patient-)individual puff profile with the measured values of the current inhalation puff and reacts accordingly as described above.

The (patient-)individual puff profile can also take into account the amount of active ingredient desired for the patient. This amount of active ingredient can, for example, be specified by a physician or a pharmacist (e.g., based on a digital prescription) and transmitted to the electronic control device and/or the data memory by means of the above-mentioned communication device.

Preferably, user data may be stored in the data memory. For example, the frequency of use and/or the number of successful inhalation puffs can be stored as a function of date and time. By reading out this data, a physician can monitor the (temporally) correct use of the inhaler and application of the active ingredient, adjust the patient’s therapy plan if necessary, and take the data into account in further diagnostics and therapy.

Furthermore, it is possible that the aforementioned user data and/or the puff profile are stored in a cloud storage via the communication device in addition to or as an alternative to the data storage. In this case, a physician can remotely monitor the use of the inhaler when accessing the data in the cloud storage.

Preferably, stored data can be transmitted to an electronic mobile device, such as a smartphone, laptop, smartwatch or tablet PC, using the communication device. In this case, the user himself can monitor the use of the inhaler using the mobile device. He can also set up appointment reminders in the mobile device if the inhaler is to be used at certain times or at recurring intervals.

Advantageously, for instance in clinical studies from phase I onwards, regular use and thus complete documentation of inhaler use can be ensured. The user can also note his personal feelings before and after using the inhaler when transferring the data to an electronic mobile device. In this way, for example, a causal relationship between certain physical reactions and the use of the inhaler or the active ingredient applied with it can be closely documented. The information thus obtained, e.g. on the tolerability of an active ingredient, can also be relevant for the doctor when treating a disease with an already approved active ingredient.

Controlled and reproducible active ingredient dispensing is a prerequisite for the use of the invention or the control of the heating current flowing through the heating element according to the invention in the field of medical application. In other words, the present invention is the basis for opening up a completely new field of application.

The invention thus solves the problem of a defined delivery of active substance or liquid per air volume with a time-varying airflow.

The invention will be explained below by means of preferred embodiments with reference to the accompanying figures. Thereby shows

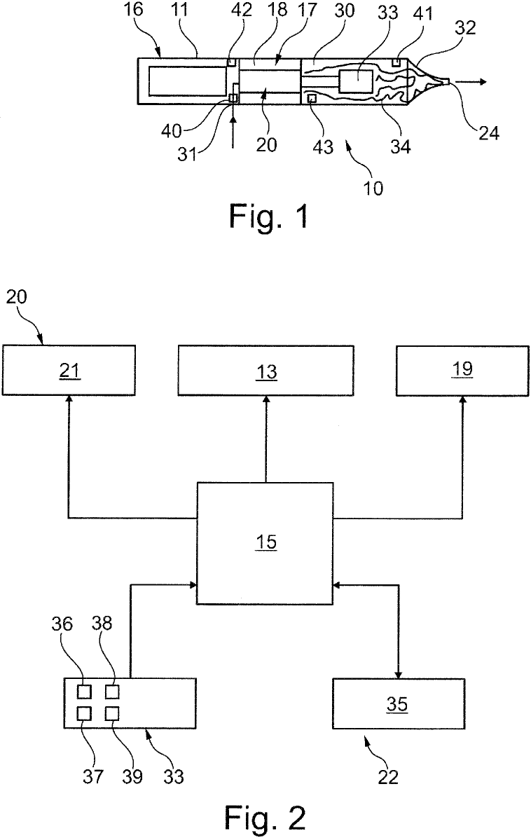

FIG. 1 a longitudinal section through an inhaler;

FIG. 2 a schematic illustration of an electronic arrangement of the inhaler with a sensor system;

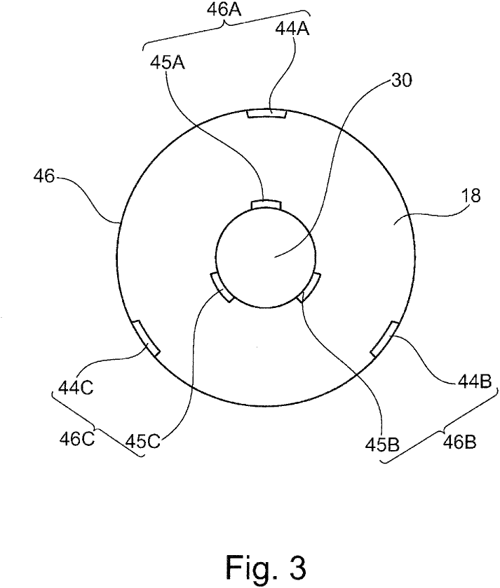

FIG. 3 a cross-sectional view of a liquid tank with an air channel centered inside;

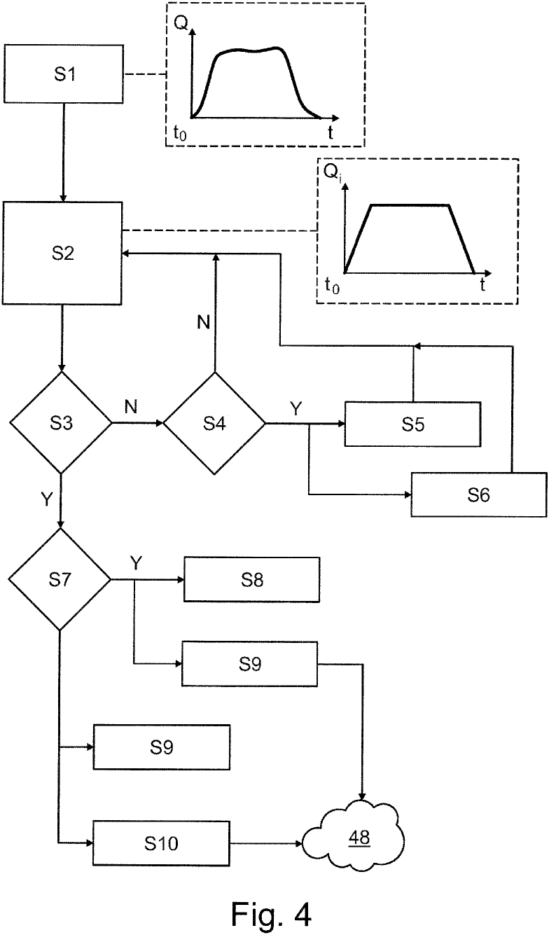

FIG. 4 a flow chart for using an inhaler; and

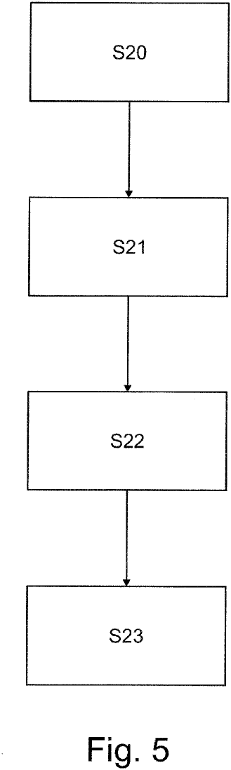

FIG. 5 a flow chart for an initialization of an inhaler.

The inhaler 10, in this case an electronic cigarette product, includes a housing 11 in which an air channel 30 is provided between at least one air inlet opening 31, and an air outlet opening 24 at a mouth portion 32 of the inhaler 10. When the consumer draws on the mouth portion 32 for inhalation, a negative pressure is thereby applied to the inhaler 10 and an air flow 34 is generated in the air channel 30.

Advantageously, the inhaler 10 comprises a liquid reservoir 18, an electrical energy storage device 14, a vaporization device 20 comprising an electrical resistance heating element 21, an electrical arrangement 22, and a sensor system 33. The liquid reservoir 18 is advantageously arranged in a consumption unit 17 of the inhaler 10. The consumption unit 17 may advantageously be in the form of a replaceable cartridge.

The electrical energy storage device 14 is advantageously arranged in a base part 16 of the inhaler 10. In particular, the energy storage device 14 may be a disposable electrochemical battery or a rechargeable electrochemical battery, for example a lithium-ion battery. Preferably, the energy storage device 14 is arranged in a portion of the inhaler 10 remote from the mouth portion 32. Advantageously, the consumption unit 17 is arranged between the energy storage device 14 and the mouth portion 32.

The electrical arrangement 22 of the inhaler 10, shown schematically in FIG. 2, comprises the electrical resistance heating element 21, a digital electronic control device 15, and an electronic data storage device 35. The electronic control device 15 is a digital data processing device and preferably comprises a microprocessor and/or a microcontroller. The electrical arrangement 22 may preferably comprise a communication device 13 and/or a signal device 19. The communication device 13 is wireless, for example based on Bluetooth, WLAN, RFID, ZigBee, optical or wired, and preferably adapted to communicate with a mobile electronic device, such as a cell phone or smartphone, an external computer and/or cloud storage. The signaling device 19 comprises optical, acoustic and/or haptic signaling elements, for example one or more LEDs, a digital display, a display, a haptic signal generator and/or an acoustic signal generator.

Parts of the electrical arrangement 22 are preferably arranged in the base part 16, for example control device 15, data memory 35, possibly communication device 13 and/or possibly signaling device 19. Parts of the electrical arrangement 22 may be arranged in the consumption unit 17, for example the heating element 21. In other embodiments, the heating element 21 is arranged in the base part 16.

Air drawn through the inlet opening 31 is directed in the air channel 30 to, through, or past the vaporization device 20. The vaporization device 20 is connected or connectable to the liquid reservoir 18, in which a liquid or a mixture of liquids is stored. The vaporizing device 20 vaporizes liquid supplied to it from the liquid reservoir 18, and adds the vaporized liquid as an aerosol/vapor to the airflow 34.

The sensor system 33 advantageously comprises a pressure or flow switch 36 arranged in the air channel 30, or flow-connected thereto, which is triggered, for example, when the pressure in the air channel 30 falls below a predefined pressure threshold. Based on a signal output by the pressure or flow switch 36, the control device 15 can determine that a consumer is drawing on the mouth portion 32 of the cigarette product 10 to inhale. The flow measurement device 37 (see below) may perform the function of the flow switch 36, or a separate flow switch 36 may be provided. The control device 15 then controls the vaporization device 20 to add liquid from the liquid reservoir 18 as an aerosol/vapor into the airflow 34.

The liquid 50 stored in the liquid reservoir 18 to be dispensed is, for example, a mixture comprising one or more of the following ingredients: 1,2-propylene glycol, glycerol, water, at least one aroma (flavor), optionally an active ingredient, for example nicotine.

The vaporization device 20 comprises at least one resistive heating element 21, and may comprise a wick element, not shown, for supplying liquid from the liquid reservoir 18 to the heating element 21. Due to ohmic resistance, current flow through the electrically conductive heating element 21 results in heating of the same and therefore vaporization of liquid in contact with the heating element 21. Vapor/aerosol generated in this way escapes from the vaporizer 20 and is mixed with the airflow 34, see FIG. 1. Depending on the liquid to be vaporized, the vaporization temperature is preferably in the range between 100° C. and 450° C., more preferably between 150° C. and 350° C., still more preferably between 190° C. and 290° C.

The data memory 35 is advantageously non-volatile and is used, for example, to store information or parameters relating to the consumption unit 17. The data memory 35 may be part of the electronic control device 15. The data memory 35 advantageously stores information on the composition of the liquid stored in the liquid reservoir 18, information on the vaporization profile, in particular for power/temperature control, data on condition monitoring or system testing, for example leak testing, data relating to copy protection and anti-counterfeiting, an ID for uniquely identifying the consumption unit 17, serial number, manufacturing date, expiration date, puff count (number of inhalation puffs by the consumer) and/or usage time.

The sensor system 33 comprises a flow measuring device 37, which is designed here as a differential pressure measuring device. The flow measuring device 37 comprises a first pressure sensor 42 (see FIG. 1), which is arranged to measure atmospheric air pressure outside the housing 11 of the inhaler 10. For example, a measurement port may be provided in the housing 11 for connecting the first pressure sensor 42 to the atmosphere in a flow conducting manner. The flow measuring device 37 further comprises a second pressure sensor 43 (see FIG. 1) arranged to measure the air pressure prevailing in the air channel 30. By knowing the cross-section of the air channel at the measurement location of the second pressure sensor 43 and the pressure difference measured between the first and second pressure sensors 42, 43, the electronic control device 15 can calculate the air volumetric flow through the inhaler 10 and determine a puff profile, i.e., the air volumetric flow over time, by repeated measurement over time.

The sensor system 33 comprises a vapor quantity sensor 38 for capturing the quantity of liquid or vapor vaporized and dispensed by the vaporizer device 20 during an inhalation puff. Here, the vapor quantity sensor 38 is an air humidity sensor. The vapor quantity sensor 38 comprises a first humidity measuring element 40 (see FIG. 1) arranged in the air channel 30 upstream of the heating element 21, for example in the area of the air inlet opening 31. The vapor quantity sensor 38 further comprises a second humidity measuring element 41 (see FIG. 1), which is arranged in the air channel 30 downstream of the heating element, for example in or in the region of the mouth portion 32. With a known amount of water in the liquid to be vaporized and calibration of the second humidity measuring element 41, the amount of liquid or vapor vaporized can be determined by the electronic control device 15 from the increase in humidity, i.e., the difference in readings from the second humidity measuring element 41 and the first humidity measuring element 40.

The liquid tank 18 is preferably elongated with a central internal air channel 30 (FIG. 3 shows a section through the liquid tank 18 transverse to the longitudinal axis L). The vaporizer device 20, not shown in FIG. 3 for the sake of clarity, vaporizes liquid supplied from the liquid tank 18 and releases it as a vapor/aerosol to the air flow passing through the internal air channel 30.

The sensor system 33 preferably comprises a level sensor 39 for capturing a residual amount of liquid in the liquid tank 18 of the inhaler 10. Here, the level sensor 39 is a capacitive sensor and comprises at least one pair of electrodes 44A, 45A; 44A, 45B; 44C, 45C (see FIG. 3) arranged on opposing walls 46, 47 of the fluid tank 18. Preferably, the electrodes 44A, 45A; 44A, 45B; 44C, 45C are formed by metallized areas, in particular metallic (longitudinal) strips, which are continuous along the longitudinal axis of the liquid tank 18. Advantageously, at least one first electrode 44A, 44B, 44C is arranged on the, for example, cylindrical outer wall 46 of the liquid tank 18 and at least one second electrode 45A, 45B, 45C is arranged on the, for example, cylindrical inner wall 46 of the liquid tank 18 (and/or on the, for example, cylindrical outer wall of the air channel 30).

The level sensor 39 preferably comprises a plurality of electrode pairs, for example at least or exactly three electrode pairs 44A, 45A; 44A, 45B; 44C, 45C. The electrode pairs 44A, 45A; 44A, 45B; 44C, 45C are advantageously mutually arranged at equal angular distances, in FIG. 3 for example at 120°, with respect to the central axis of the liquid tank 18. The first electrodes 44A, 44B, 44C and/or the second electrodes 45A, 45B, 45C may each be connected to form a continuous electrode 44 or 45, respectively.

Each pair of electrodes 44A, 45A; 44A, 45B; 44C, 45C forms a capacitor 46A, 46B, 46C with the (residual) liquid in the tank as dielectric. The capacitance of each capacitor 46A, 46B, 46C is measured continuously. The measured capacitance of each capacitor 46A, 46B, 46C is proportional to the liquid level between the electrodes 44A, 45A; 44A, 45B; 44C, 45C. The arrangement and number of capacitors 46A, 46B, 46C can be used to determine the liquid level in the liquid tank 18 in any spatial orientation of the inhaler 10 or cartridge or consumption unit 17.

From the determined liquid level in the liquid tank, the residual amount of liquid in the liquid tank 18 can be determined. From the determined remaining amount of liquid, for example, a prediction can be made as to when a change of the consumption unit 17 (cartridge change) is likely to be required, based on the previous usage profile of the current user.

In the following, a preferred method for controlling the inhaler 10 is explained with reference to FIG. 4.

In step S1, the volumetric flow Q = dV/dt (see diagram on the right) of the air flow (or air/steam flow) passing through the air channel 30 is measured over time by means of the flow measuring device 37. The volumetric flow Q is measured at the respective current time ta and preferably the resulting puff profile Q(t) over an entire inhalation puff. The volume flow Q plotted over time t for an entire inhalation puff of the user results in a puff profile Q(t), as shown, for example, in the diagram to the right of step S1.

An ideal puff profile Qi(t) is stored in the electronic data memory 35 of the inhaler 10, see diagram to the right of step S2.

In step S2, the electronic control device 15 performs a comparison analysis. This involves a comparison of the current volumetric flow Q(t=ta) measured in step S1 with a corresponding ideal value Qi(t=ta) resulting from the stored puff profile Qi(t), and preferably a comparison of the puff profile Q(t) measured in step S1 (in particular in the period t0 corresponding to the start of the puff to ta) with the stored ideal puff profile Qi(t). The comparative analysis further comprises determining a deviation of the actual volume flow Q(t=ta) measured in step S1 from the corresponding ideal value Qi(t=ta), and preferably determining a deviation of the puff profile Q(t) measured in step S1 from the ideal puff profile Qi(t). In step S2, the measured actual volume flow Q(t=ta) can be a current value corresponding to a single measured value, or a value averaged over a plurality of measured values Q(t=ta1), Q(t=ta2). ....

In the following step S3 it is checked whether the current inhalation puff has ended. If the current inhalation puff has not ended (N), in step S4 it is checked whether the deviation of the measured current or instantaneous volume flow Q(t=ta) from the corresponding ideal value Qi(t=ta) determined in step S2 is above a threshold value stored in the data memory 35.

If the current deviation considered in step S4 is above the stored threshold value (Y), in step S5 the active ingredient dispensing into the airflow is adjusted, preferably based on the degree of current deviation, by controlling the vaporization device 20, in particular by changing the heating current flowing through the heating element 21 and/or the heating duration of the heating element 21.

In addition or alternatively to step S5, in step S6 an indication of the incorrect or non-ideal current use of the inhaler 10 can be initiated to the user via at least one signal element of the signal device 19 by the electronic control device 15, e.g. the indication of an incorrect air quantity and/or active ingredient quantity.

Following step S5 and/or S6, or if the current deviation considered in step S4 is below the stored threshold value (N), step S2 is performed again. This feedback loop is performed until it is determined in the step S3 that the inhalation puff has ended (Y).

Once it is determined in step S3 that the inhalation puff has ended (Y), it is preferably checked in step S7 whether a deviation of the puff profile Q(t) measured in step S1 from the ideal puff profile Qi(t), in each case over the entire inhalation puff (total deviation), is above a threshold value stored in the data memory 35.

If in step S7 it is determined that the total deviation is above the stored threshold value (Y), in a step S8 an indication of the incorrect or non-ideal use of the inhalation puff 10 can be initiated to the user via at least one signal element of the signal device 19 by the electronic control device 15, e.g. the indication of an incorrect amount of air and/or amount of active ingredient via the relevant inhalation puff.

In addition or alternatively to step S8, in a step S9, a transmission of the data from the comparative analysis of step S2 may be sent, for example by wireless communication, to a remote receiver 48, for example a cloud storage, for further analysis.

If in step S7 it is determined that the total deviation is below the stored threshold (N), in a step S9 an indication of the successful use of the inhaler 10 to the user via at least one signal element of the signaling device 19 may be initiated by the electronic control device 15, for example the indication of a correct amount of air and/or amount of active ingredient via the relevant inhalation puff.

In addition or alternatively to step S9, in a step S10, a transmission of the data from the comparative analysis of step S2 may be sent, for example by wireless communication, to a remote receiver 48, for example a cloud storage, for further analysis.

An initialization procedure for the inhaler is explained below with reference to FIG. 5.

In step S20, puff profiles over a plurality of inhalation puffs, preferably without active ingredient dispensing, are measured by the flow measurement device 37 and generated by the electronic control device 15.

In step S21, the electronic control device 15 performs a comparison of the measured puff profiles with a theoretical puff profile, which is advantageously stored in the data memory 35.

In the step S22, the electronic control device 15 performs a fitting of the theoretical puff profile to the measured puff profiles.

In step S23, the fitted puff profile is stored by the electronic control device 15 in the data memory 35 as an ideal (user) puff profile in order to be available for comparisons or comparative analyses during the use phase.

1. An inhaler comprising a housing, an air channel extending between at least one air inlet opening and a suction opening (24) in the housing, a dispensing element for nebulizing or vaporizing liquid supplied to the dispensing element for admixture to air flowing in the air channel, an electronic control device, an electronic data memory and a sensor system comprising a flow measuring device for measuring the volumetric and/or mass flow of the airflow flowing through the air channel, wherein the electronic control device is adapted to capture a plurality of airflow measurement values over at least a portion of the duration of an inhalation puff by means of the flow measurement device, to compare the plurality of airflow measurement values to a puff profile stored in the data memory, and to output a control signal based on the comparison of the plurality of airflow measurement values to the stored puff profile.

2. The inhaler according to claim 1, wherein a defined deviation of the airflow measurement values from the stored puff profile, the control signal triggers a suitable action.

3. The inhaler according to claim 2, wherein the triggered action is one or more of the following:
adjusting the control of the dispensing element for the amount of liquid to be vaporized or nebulized; and
signaling corresponding information to a user by means of a signaling device.

4. The inhaler according to claim 1, wherein the flow measuring device is a differential pressure measuring device or a hot-wire measuring device.

5. The inhaler according to claim 4, wherein the differential pressure measuring device comprises a first pressure sensor for measuring the atmospheric air pressure and a second pressure sensor for measuring the air pressure prevailing in the air channel.

6. The inhaler according to claim 1, wherein the sensor system comprises a vapor quantity sensor for capturing the quantity of liquid delivered by a vaporization device during an inhalation puff.

7. The inhaler according to claim 6, wherein the vapor quantity sensor is a humidity sensor.

8. The inhaler according to claim 7, wherein the humidity sensor comprises a first humidity sensing element arranged in the air channel upstream of the dispensing element and a second humidity sensing element arranged in the air channel downstream of the dispensing element.

9. The inhaler according to claim 6, wherein the liquid quantity sensor is an optical absorption sensor.

10. The inhaler according to claim 6, wherein the electronic control device is adapted to compensate for a changing water content in the liquid during the emptying of the liquid tank by determining the residual amount of liquid in the liquid tank by means of a level sensor or estimating by counting the puffs in connection with the measurement of the puff duration.

11. The inhaler according to claim 6, wherein the electronic control device is adapted to calculate the vaporized amount of active ingredient from the determined vaporized amount of liquid.

12. The inhaler according to claim 1, wherein the sensor system comprises a level sensor for capturing a residual liquid quantity in a liquid tank of the inhaler.

13. The inhaler according to claim 12, wherein the level sensor is a capacitive sensor.

14. The inhaler according to claim 12, wherein the electronic control device is arranged to determine a duration until a cartridge change is required based on the amount of residual liquid measured by means of the level sensor.

15. The inhaler according to claim 1, wherein the electronic control device is adapted to perform an initialization procedure prior to actual use comprising the following steps:
measuring a plurality of airflow measurement values over one or more inhalation puffs using the flow measurement device;
comparison of the airflow measurement values with, for example, a theoretical puff profile stored in the data memory;
determining a user-specific ideal puff profile from the comparison of the airflow measurement values with the theoretical puff profile; and
storing the user-individual ideal puff profile in the data memory.
